(19) United States
(12) Patent Application Publication
(10) Pub. No.: US 2013/0252904 A1
(43) Pub. Date: Sep. 26, 2013

(54) COMPOSITIONS AND METHODS FOR TREATING CANCER

(71) Applicant: ENDOCYTE, INC., West Lafayette, IN (US)

(72) Inventors: Christopher P. Leamon, West Lafayette, IN (US); Iontcho R. Vlahov, West Lafayette, IN (US)

(73) Assignee: Endocyte, Inc., West lafayette, IN (US)

(21) Appl. No.: 13/779,537

(22) Filed: Feb. 27, 2013

Related U.S. Application Data
(60) Provisional application No. 61/605,137, filed on Feb. 29, 2012.

(51) Int. Cl. A61K 47/48

(52) U.S. Cl. CPC A61K 47/48107
USPC 514/19.3; 530/328

(57) ABSTRACT

The invention relates to compounds, compositions, kits, and methods for the treatment of a cancer. In particular, the invention relates to compounds, compositions, or kits comprising a folate-vinca conjugate, and methods for the treatment of a cancer with a folate-vinca conjugate.

### RELATED APPLICATIONS

This application claims the benefit of U.S. Provisional Application No. 61/605,137, filed Feb. 29, 2012, the content of which is incorporated by reference.

### TECHNICAL FIELD

The invention relates to compositions and methods for the treatment of a cancer. In particular, the invention relates to compositions and methods for the treatment of a cancer with a particular folate-vinca conjugate.

### BACKGROUND AND SUMMARY

Despite the fact that there have been significant developments in anti-cancer technology, such as radiotherapy, chemotherapy and hormone therapy, cancer still remains the second leading cause of death following heart disease in the United States. Most often, cancer is treated with chemotherapy utilizing highly potent drugs, such as platinum compounds, mitomycin, paclitaxel and camptothecin. In many cases, these chemotherapeutic agents show a dose response effect, and cell killing is proportional to the drug dose. A highly aggressive style of dosing is thus necessary to eradicate the cancer. However, high-dose chemotherapy is hindered by poor selectivity for cancer cells and severe toxicity to normal cells. This lack of tumor-specific treatment is one of the many hurdles that needs to be overcome by current chemotherapies.

One solution to current chemotherapy limitations would be to deliver a biologically effective concentration of anti-cancer agents to the tumor tissues with very high specificity. To reach this goal, much effort has been undertaken to develop tumor-selective drugs by conjugating anti-cancer drugs to such ligands as hormones, antibodies, or vitamins. For example, the low molecular weight vitamin compound, folate, is useful as a tumor-targeting agent.

Folate is a member of the B family of vitamins and plays an essential role in cell survival by participating in the biosynthesis of nucleic acids and amino acids. This essential vitamin is also a high affinity ligand that enhances the specificity of conjugated anti-cancer drugs by targeting folate receptor (FR)-positive cancer cells. The FR, a tumor-associated glycosylphosphatidylinositol anchored protein, can actively internalize bound folates and folate conjugated compounds via receptor-mediated endocytosis. It has been found that the FR is upregulated in more than 90% of non-mucinous ovarian carcinomas. The FR is also found at high to moderate levels in kidney, brain, lung, and breast carcinomas while it occurs at low levels in most normal tissues. The FR density also appears to increase as the stage of the cancer becomes more advanced.

Accordingly, the present invention relates to the development of folate-targeted therapeutics to treat cancer. The folate conjugate described herein can be used to treat cancer by targeting cancer cells that overexpress the folate receptor. In one embodiment, a method of treatment of a cancer is provided, comprising administering EC0905 to a patient in need thereof. In another embodiment, a composition comprising EC0905 is described. In yet another embodiment, a pharmaceutical composition comprising EC0905 is described. In yet another embodiment, the use of EC0905 for the treatment of a folate receptor expressing cancer is described. EC0905 is a compound of the formula:

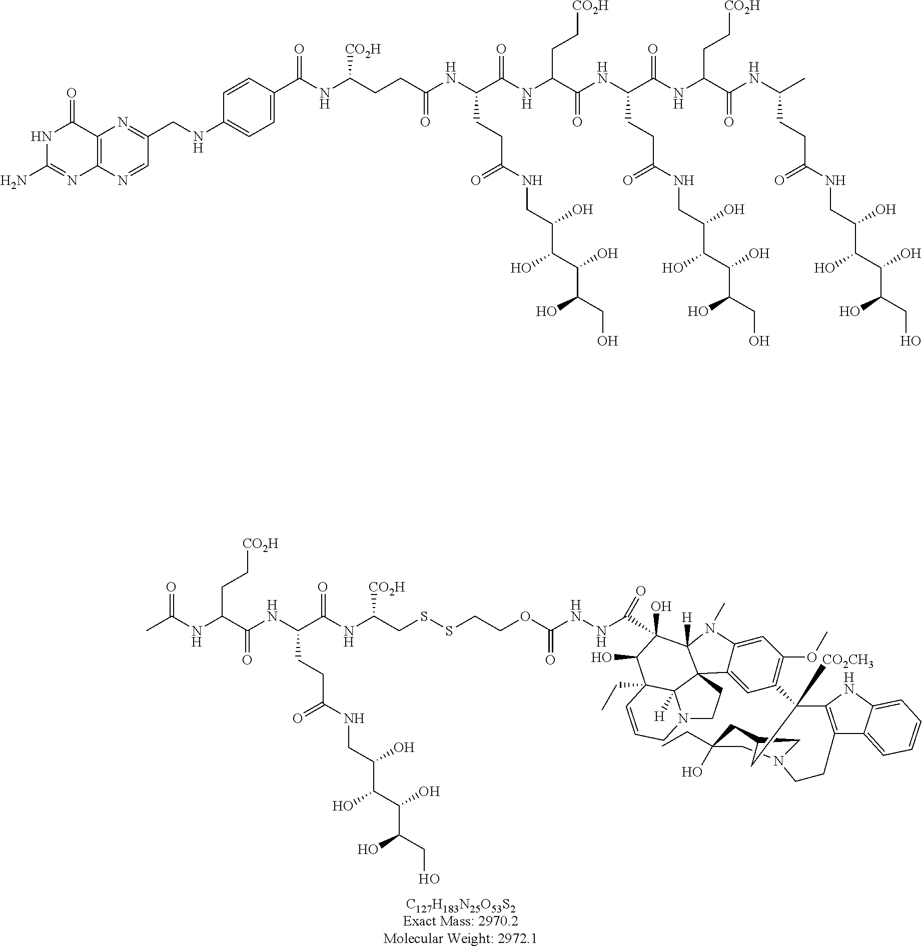

As used herein, in the context of a compound, composition, a pharmaceutical composition, or a kit, the term “EC0905” means the chemotherapeutic agent, the structure of which is shown above, or a pharmaceutically acceptable salt thereof. The chemotherapeutic agent may be present in solution or suspension in an ionized form, including a protonated form. EC0905 can be synthesized, for example, by the method described in Example 2. “EC0905” is used interchangeably with the term “conjugate” herein.

In one example embodiment, the EC0905 is in a composition and the composition further comprises a pharmaceutically acceptable carrier. In one example embodiment, the pharmaceutically acceptable carrier comprises a liquid. In some embodiments, the liquid is saline, glucose, an alcohol, a glycol, an ester, an amide, or a combination thereof. In some embodiments, the pharmaceutically acceptable carrier comprises a lyophilizate. In some embodiments, the lyophilizate is a reconstitutable lyophilizate.

In other embodiments, the compound or the composition is an inhalation dosage form, an oral dosage form, or a parenteral dosage form. In one example embodiment, the parenteral dosage form is an intradermal dosage form, a subcutaneous dosage form, an intramuscular dosage form, an intraperitoneal dosage form, an intravenous dosage form, or an intrathecal dosage form.

In another example embodiment, the compound or the composition is in the form of a solid. In some embodiments, the purity of the compound is at least 90%, 95%, 98%, or 99% based on weight percent.

In one example embodiment, a kit is provided comprising a sterile vial, a compound or composition, and instructions describing use of the compound or composition for treating a patient with cancer. In some embodiments, the compound or composition is EC0905. In some embodiments, the EC0905 in the kit is in the form of a reconstitutable lyophilizate. In some embodiments, the dose of the EC0905 compound in the kit is in the range of 1 to 5 μg/kg of patient body weight. In other embodiments, the dose of the compound in the kit is in the range of 1 to 3 μg/kg of patient body weight. In some embodiments, the purity of the compound in the kit is at least 90%, 95%, 98%, or 99% based on weight percent.

In one embodiment, the EC0905 in the kit is in a composition, and the composition further comprises a pharmaceutically acceptable carrier. In one example embodiment, the pharmaceutically acceptable carrier comprises a liquid. In some embodiments, the liquid carrier is saline, glucose, an alcohol, a glycol, an ester, an amide, or a combination thereof.

In one embodiment, the EC0905 compound or composition in the kit is in a parenteral dosage form. In one example embodiment, the parenteral dosage form is an intradermal dosage form, a subcutaneous dosage form, an intramuscular dosage form, an intraperitoneal dosage form, an intravenous dosage form, or an intrathecal dosage form.

Another embodiment entails the use of a compound of the formula

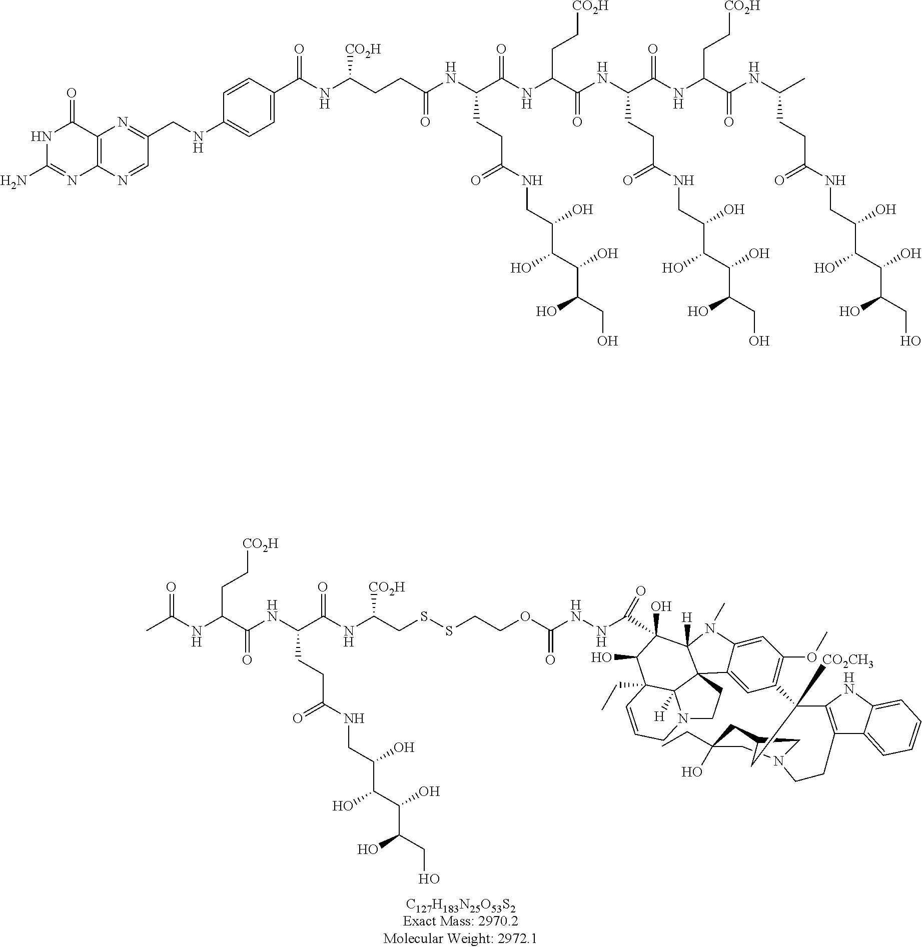

for the manufacture of a medicament for treating cancer.

In one example embodiment, the EC0905 used is in a composition, and use of the composition further comprises a pharmaceutically acceptable carrier. In one example embodiment, the use of the pharmaceutically acceptable carrier comprises a liquid. In some embodiments, the liquid carrier used is saline, glucose, an alcohol, a glycol, an ester, an amide, or a combination thereof.

In other embodiments, the use of the composition is an inhalation dosage form, an oral dosage form, or a parenteral dosage form. In some example embodiments, the parenteral dosage form used is an intradermal dosage form, a subcutaneous dosage form, an intramuscular dosage form, an intraperitoneal dosage form, an intravenous dosage form, or an intrathecal dosage form.

In another example embodiment, the use of the compound or composition is in the form of a solid. In one example embodiment, the use of the compound or composition is in the form of a suspension. In some embodiments, the purity of the compound is at least 90%, 95%, 98%, or 99% based on weight percent.

In some example embodiments, the use of the compound or composition is in the form of a lyophilizate. In some embodiments, the lyophilizate is a reconstitutable lyophilizate.

A method of treatment of a cancer is disclosed, the method comprising administering to a patient a therapeutically effective amount of a compound of the formula

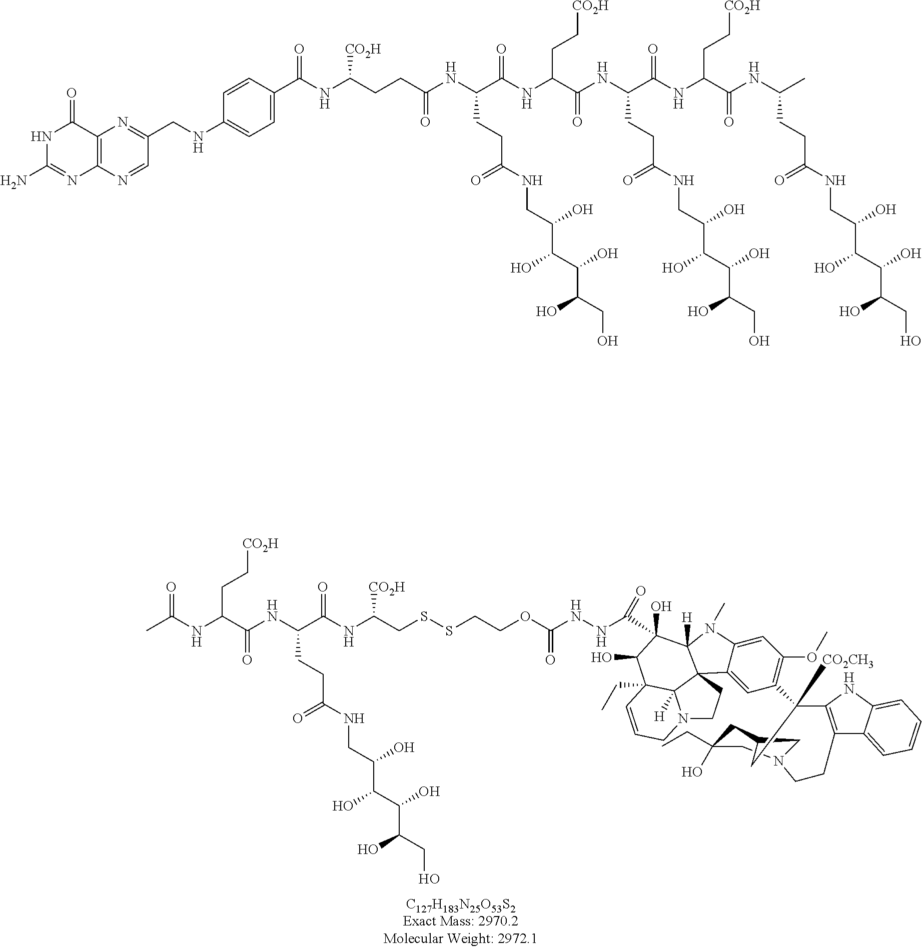

In some example embodiments, the compound is in a composition and the composition further comprises a pharmaceutically acceptable carrier. The composition may be an inhalation dosage form, an oral dosage form, or a parenteral dosage form. In currently preferred embodiments, the compound or the composition is a parenteral dosage form. The parenteral dosage form may be an intradermal dosage form, a subcutaneous dosage form, an intramuscular dosage form, an intraperitoneal dosage form, an intravenous dosage form, or an intrathecal dosage form.

In some example embodiments, the pharmaceutically acceptable carrier comprises a liquid. The liquid carrier is saline, glucose, an alcohol, a glycol, an ester, an amide, or a combination thereof. In some example embodiments, the compound or the composition is in the form of a solid. The purity of the compound may be at least 90, 95, 98, or 99% based on weight percent. In some example embodiments, the compound is a pharmaceutically acceptable salt of EC0905. In certain embodiments, the pharmaceutically acceptable salt of EC0905 is a sodium salt.

### BRIEF DESCRIPTION OF THE DRAWINGS

FIG. 1 shows the EC0905 conjugate structure.

FIG. 2 shows the in vitro activity of EC0905. The open circles are EC0905 and the closed circles are EC0905 plus excess competing EC17.

FIG. 3 shows the effect of EC0905 on the volume of subcutaneous KB tumors in nu/nu mice as a function of days post tumor cell inoculation (“PTI”). The closed squares are the KB control samples. The closed circles are data with mice treated with EC0905, 2 μmol/kg, TIW, twice per week.

FIG. 4 shows the effect of EC0905 on the body weights of nu/nu mice as a function of days post tumor cell inoculation (“PTI”). The closed squares are the KB control samples. The closed circles are data with mice treated with EC0905, 2 μmol/kg, TIW, twice per week.

### DETAILED DESCRIPTION OF ILLUSTRATIVE EMBODIMENTS

A compound is disclosed of the formula

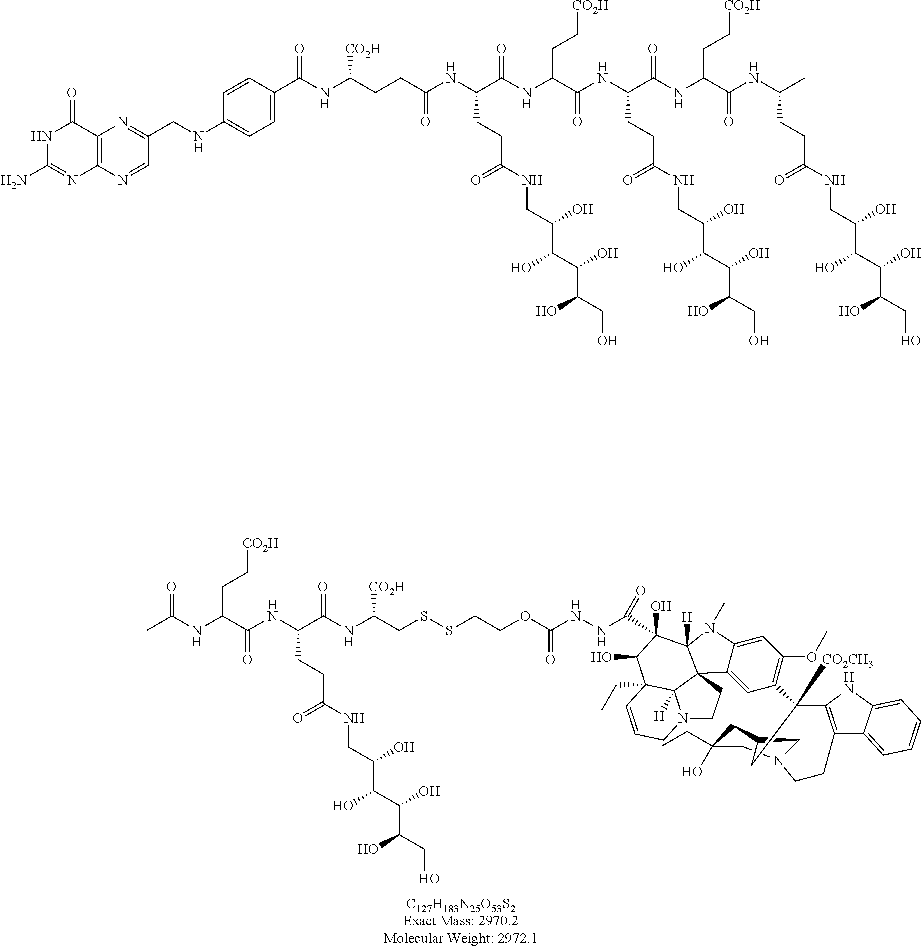

In some example embodiments, the EC0905 compound is in a composition and the composition further comprises a pharmaceutically acceptable carrier. The composition may be an inhalation dosage form, an oral dosage form, or a parenteral dosage form. In currently preferred embodiments, the compound or the composition is in a parenteral dosage form. The parenteral dosage form may be an intradermal dosage form, a subcutaneous dosage form, an intramuscular dosage form, an intraperitoneal dosage form, an intravenous dosage form, or an intrathecal dosage form.

In some example embodiments, the pharmaceutically acceptable carrier comprises a liquid. The liquid carrier is saline, glucose, an alcohol, a glycol, an ester, an amide, or a combination thereof. In some example embodiments, the compound or the composition is in the form of a solid. The purity of the compound may be at least 90, 95, 98, or 99% based on weight percent. In some example embodiments, the compound is a pharmaceutically acceptable salt of EC0905. In certain embodiments, the pharmaceutically acceptable salt of EC0905 is a sodium salt.

A kit is disclosed comprising a sterile vial, the EC0905 compound or composition, and instructions for use describing use of the compound or composition for treating a patient with cancer. In some embodiments, the compound or composition of the kit is in the form of a reconstitutable lyophilizate. In some embodiments, the dose of the compound included in the kit is in the range of 1 to 5 μg/kg of patient body weight. In other embodiments, the dose of the compound included in the kit is in the range of 1 to 3 μg/kg of patient body weight.

In some example embodiments, the EC0905 compound included in the kit is in a composition and the composition further comprises a pharmaceutically acceptable carrier. The composition included in the kit may be an inhalation dosage form, an oral dosage form, or a parenteral dosage form. In currently preferred embodiments, the compound or the composition included in the kit is in a parenteral dosage form. The parenteral dosage form may be an intradermal dosage form, a subcutaneous dosage form, an intramuscular dosage form, an intraperitoneal dosage form, an intravenous dosage form, or an intrathecal dosage form.

In some example embodiments, the pharmaceutically acceptable carrier comprises a liquid. The liquid carrier is saline, glucose, an alcohol, a glycol, an ester, an amide, or a combination thereof. In some example embodiments, the compound or the composition included in the kit is in the form of a solid. The purity of the compound included in the kit may be at least 90, 95, 98, or 99% based on weight percent. In some example embodiments, the compound included in the kit is a pharmaceutically acceptable salt of EC0905. In certain embodiments, the pharmaceutically acceptable salt of EC0905 is a sodium salt.

Another embodiment entails the use of a compound of the formula

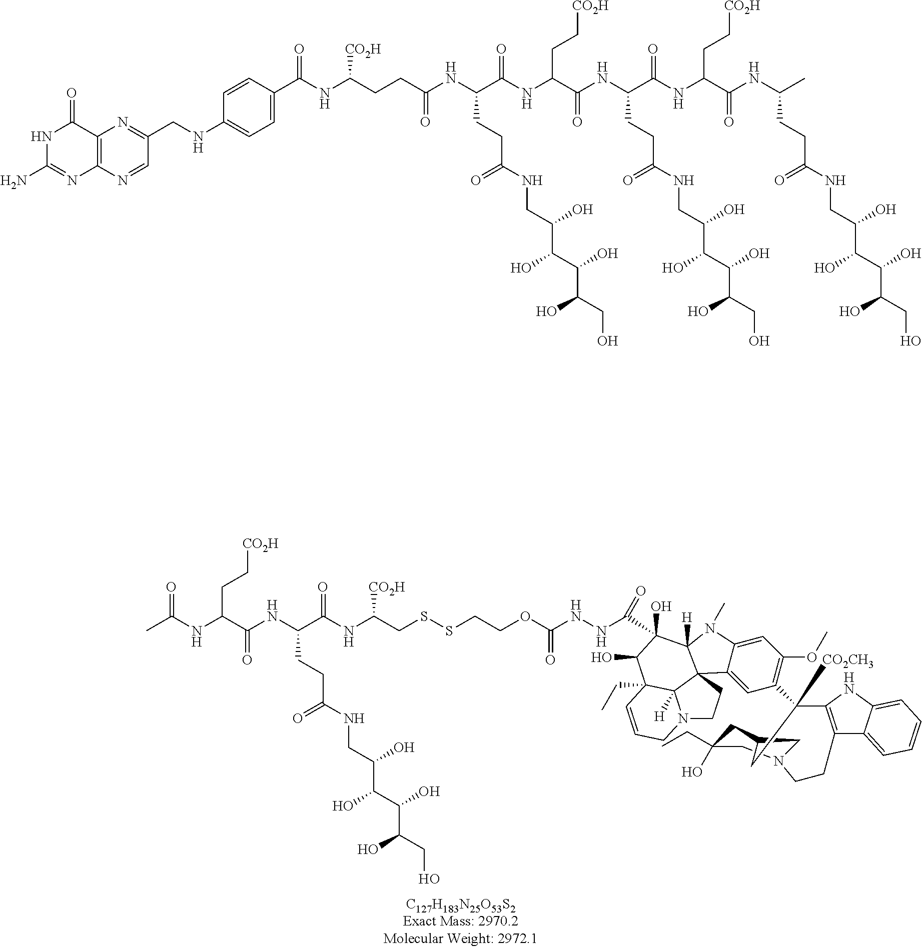

for the manufacture of a medicament for treating cancer.

In some example embodiments, the compound used to treat the cancer is in the form of a composition. In some example embodiments, the composition further comprises a pharmaceutically acceptable carrier. In some example embodiments, the composition is an inhalation dosage form, an oral dosage form, or a parenteral dosage form. In preferred embodiments, the composition used to treat the cancer is a parenteral dosage form, and the parenteral dosage form is an intradermal dosage form, a subcutaneous dosage form, an intramuscular dosage form, an intraperitoneal dosage form, an intravenous dosage form, or an intrathecal dosage form.

In some example embodiments, the pharmaceutically acceptable carrier used to treat the cancer comprises a liquid. The liquid carrier is saline, glucose, an alcohol, a glycol, an ester, an amide, or a combination thereof. In some example embodiments, the compound used to treat the cancer is in the form of a solid. Alternatively, the compound may be in the form of a suspension. In some example embodiments, the purity of the compound used to treat the cancer is at least 90% based on weight percent. Alternatively, the purity of the compound used to treat the cancer may be at least 95%, 98%, or 99% based on weight percent. In some example embodiments, the compound used is a pharmaceutically acceptable salt of EC0905. In certain embodiments, the pharmaceutically acceptable salt of EC0905 is a sodium salt.

A method of treatment of a cancer is disclosed, comprising administering to a patient a therapeutically effective amount of a compound of the formula:

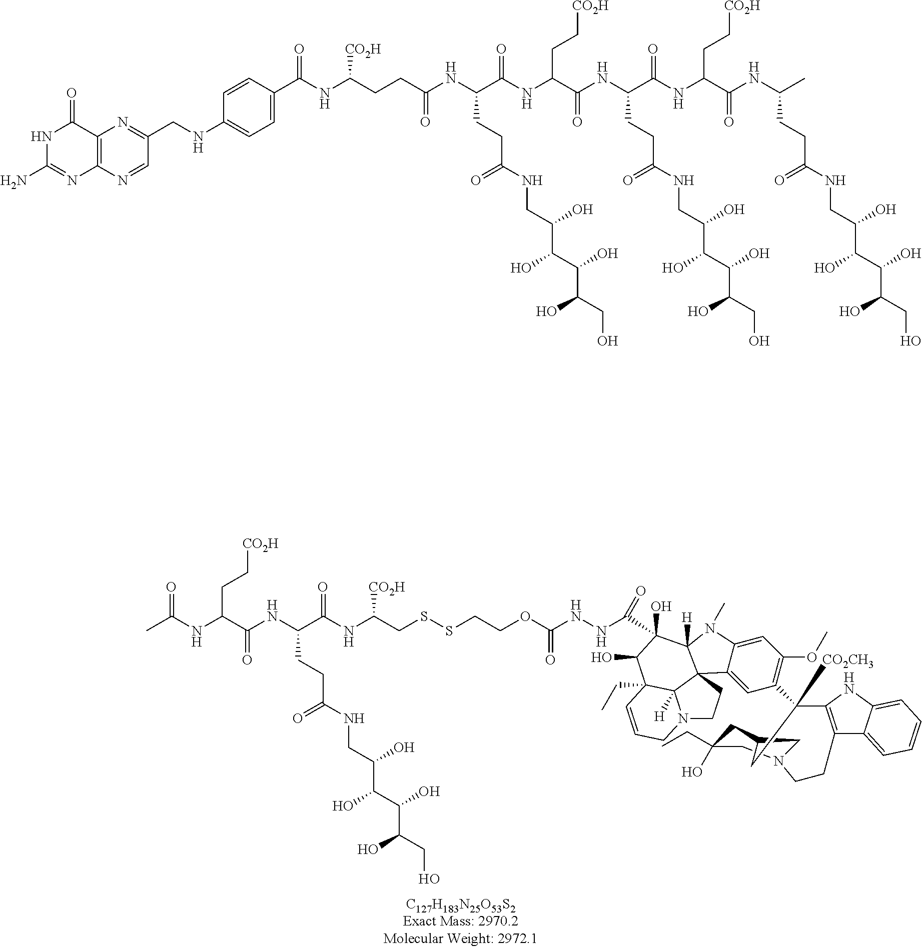

In one embodiment there is provided a method of treatment of a folate receptor expressing cancer in a patient in need thereof comprising the step of administering a therapeutically effective amount of EC0905 to the patient. A further embodiment is the use of EC0905 for the manufacture of a medicament for the treatment of a folate receptor expressing cancer in a patient. Another embodiment is EC0905 for use in treating a patient with a folate receptor expressing cancer. In each of these embodiments, a folate imaging agent conjugate as described in U.S. Pat. No. 7,862,798, incorporated herein by reference, can be used to select patients for therapy. Surface-expressed vitamin receptors, such as the high-affinity folate receptor, are overexpressed on cancer cells. Epithelial cancers have been reported to express elevated levels of the folate receptor. Accordingly, the conjugate described herein can be used to treat a variety of cancers (i.e., tumor cell types).

The method described herein can be used for both human clinical medicine and animals. Thus, the patient treated using the method herein described can be human or can be a laboratory, agricultural, domestic, or wild animal. Thus, the methods described herein are useful for treating humans, laboratory animals such rodents (e.g., mice, rats, hamsters, etc.), rabbits, monkeys, chimpanzees, domestic animals, agricultural animals such as cows, horses, pigs, sheep, goats, ostriches, and wild animals in captivity such as bears, pandas, lions, tigers, leopards, elephants, zebras, giraffes, gorillas, dolphins, sea lions, or whales.

In other embodiments of the compound, methods, uses, compositions, pharmaceutical compositions, or kits described herein, pharmaceutically acceptable salts of the conjugate described herein are described. Pharmaceutically acceptable salts of the conjugate described herein include the acid addition salts and salts made with bases.

Suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable salts made with bases of the conjugate described herein are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemi-salts of acids and bases may also be formed, for example, hemi-sulphate and hemi-calcium salts.

In other embodiments of the compound, methods, uses, compositions, pharmaceutical compositions, or kits described herein, pharmaceutically acceptable hydrates or solvates of the conjugate described herein are described. Solvated forms, or solvates, are conjugates containing either stoichiometric or nonstoichiometric amounts of solvent molecules. If the contained solvent is water, the solvates are also commonly known as hydrates.

In one embodiment, the conjugate described herein may be administered as a formulation in association with one or more pharmaceutically acceptable carriers. The carriers can be excipients. The choice of carrier will to a large extent depend on factors such as the particular mode of administration, the effect of the carrier on solubility and stability, and the nature of the dosage form. Pharmaceutical compositions suitable for the delivery of the conjugate or additional chemotherapeutic agents to be administered with the conjugate and methods for their preparation will be readily apparent to those skilled in the art. Such compositions and methods for their preparation may be found, for example, in Remington: The Science & Practice of Pharmacy, 21st Edition (Lippincott Williams & Wilkins, 2005), incorporated herein by reference.

In one embodiment, a pharmaceutically acceptable carrier may be any and all solvents, dispersion media, coatings, antibacterial and antifungal agents, isotonic and absorption delaying agents, and the like, or combinations thereof, that are physiologically compatible. In some embodiments, the carrier is suitable for parenteral administration. Pharmaceutically acceptable carriers include sterile aqueous solutions or dispersions and sterile powders for the preparation of sterile injectable solutions or dispersions. Supplementary active compounds can also be incorporated into compositions of the invention.

In various embodiments, liquid formulations may include suspensions and solutions. Such formulations may comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid.

In one embodiment, an aqueous suspension may contain the active materials in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or phydroxybenzoate; or one or more coloring agents.

In one illustrative embodiment, dispersible powders and granules suitable for preparation of an aqueous suspension by the addition of water provide the active ingredient in admixture with a dispersing or wetting agent, suspending agent and one or more preservatives. Additional excipients, for example, coloring agents, may also be present.

Suitable emulsifying agents may be naturally-occurring gums, for example, gum acacia or gum tragacanth; naturally-occurring phosphatides, for example, soybean lecithin; and esters including partial esters derived from fatty acids and hexitol anhydrides, for example, sorbitan monooleate, and condensation products of the said partial esters with ethylene oxide, for example, polyoxyethylene sorbitan monooleate.

In other embodiments, isotonic agents, for example, sugars, polyalcohols such as mannitol, sorbitol, or sodium chloride can be included in the composition. Prolonged absorption of the injectable compositions can be brought about by including in the composition an agent which delays absorption, for example, monostearate salts and gelatin.

In one aspect, a conjugate or additional chemotherapeutic agent as described herein may be administered directly into the blood stream, into muscle, or into an internal organ. Suitable routes for such parenteral administration include intravenous, intraarterial, intraperitoneal, inhalation, intrathecal, epidural, intracerebroventricular, intraurethral, intrasternal, intracranial, intratumoral, intramuscular and subcutaneous delivery. Suitable means for parenteral administration include needle (including microneedle) injectors, needle-free injectors and infusion techniques.

Examples of parenteral dosage forms include aqueous solutions of the active agent, in an isotonic saline, glucose (e.g., 5% glucose solutions), or other well-known pharmaceutically acceptable liquid carriers such as liquid alcohols, glycols, esters, and amides. The parenteral dosage form can be in the form of a reconstitutable lyophilizate comprising the dose of the conjugate. In one aspect of the present embodiment, any of a number of prolonged release dosage forms known in the art can be administered such as, for example, by using biodegradable carbohydrate matrices, or a slow pump (e.g., an osmotic pump).

In one illustrative aspect, parenteral formulations are typically aqueous solutions which may contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water. In other embodiments, any of the liquid formulations described herein may be adapted for parenteral administration of the conjugates or additional chemotherapeutic agents described herein. The preparation of parenteral formulations under sterile conditions, for example, by lyophilization under sterile conditions, may readily be accomplished using standard pharmaceutical techniques well-known to those skilled in the art. In one embodiment, EC0905 can be present in the form of a reconstitutable lyophilizate. In one embodiment, the solubility of a conjugate used in the preparation of a parenteral formulation may be increased by the use of appropriate formulation techniques, such as the incorporation of solubility-enhancing agents.

In various embodiments, formulations for parenteral administration may be formulated for immediate and/or modified release. In one illustrative aspect, active agents of the invention may be administered in a time release formulation, for example in a composition which includes a slow release polymer. The active compounds can be prepared with carriers that will protect the compound against rapid release, such as a controlled release formulations, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art. In another embodiment, the conjugates or compositions comprising the conjugates may be continuously administered, where appropriate.

One embodiment of the invention is a solid pharmaceutical composition comprising EC0905 and a bulking agent. As noted above the term EC0905 means the compound, or a pharmaceutically acceptable salt thereof; and the compound may be present in an ionized form, including a protonated form. It will be appreciated that the pH of a solution of EC0905 may be adjusted, for example by the use of 1.0 N hydrochloric acid or 1.0 N sodium hydroxide solution, and removal of water from the solution will afford a corresponding pharmaceutically acceptable salt.

Another embodiment of the solid pharmaceutical compositions described herein is an embodiment further comprising an excipient. In one embodiment the excipient comprises a buffer. In one embodiment, the pH of the buffer is about 5.0 to about 8.0. In another embodiment, the pH of the buffer is about 5.7 to about 6.6. In another embodiment, the pH of the buffer is about 6.0 to about 6.6. In another embodiment, the pH of the buffer is about 6.4±0.2.

The buffer may be any acceptable buffer for the indicated pH range and physiological compatibility. In addition a buffer may additionally act as a stabilizer. In one embodiment, the buffer comprises an ascorbate, sorbate, formate, lactate, fumarate, tartrate, glutamate, acetate, citrate, gluconate, histidine, malate, phosphate or succinate buffer. In one embodiment, the concentration of the above buffer is about 20 mM to 150 mM.

As an embodiment of the invention, there is described a lyophilized solid pharmaceutical composition comprising EC0905 which is made by a process comprising lyophilizing a liquid composition comprising EC0905, a bulking agent, optionally a buffer and an aqueous solvent.

Also contemplated herein are kits comprising the conjugate described herein. In another embodiment, a kit comprising a sterile vial, the composition of any one of the preceding embodiments, and instructions for use describing use of the composition for treating a patient with cancer is described. In some embodiments, the composition of the kit of the preceding embodiment is in the form of a reconstitutable lyophlizate is described. In another embodiment, the dose of the conjugate in the kit is in the range of 1 to 5 μg/kg. In other embodiments, the dose of the conjugate in the kit is in the range of 1 to 3 μg/kg. In another embodiment, the purity of the compound is at least 90%, 95%, 98%, or 99% based on weight percent.

In one embodiment, sterile injectable solutions can be prepared by incorporating the active agent in the required amount in an appropriate solvent with one or a combination of ingredients described above, as required, followed by sterile filtration. Typically, dispersions are prepared by incorporating the active compound into a sterile vehicle which contains a dispersion medium and any additional ingredients from those described above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof, or the ingredients may be sterile-filtered together.

The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. In one embodiment, the proper fluidity can be maintained, for example, by the use of a coating such as lecithin, by the maintenance of the required particle size in the case of dispersion and by the use of surfactants.

Any effective regimen for administering EC0905 can be used. For example, EC0905 can be administered as a single dose, or can be divided and administered as a multiple dose daily regimen. Further, a staggered regimen, for example, one to five days per week can be used as an alternative to daily treatment, and for the purpose of the compounds, compositions, kits, methods, and uses described herein, such intermittent or staggered daily regimen is considered to be equivalent to every day treatment and is contemplated. In one illustrative embodiment the patient is treated with multiple injections of EC0905 to eliminate the tumor(s). In one embodiment, the patient is injected multiple times (preferably about 2 up to about 50 times) with EC0905, for example, at 12-72 hour intervals or at 48-72 hour intervals. Additional injections of EC0905 can be administered to the patient at an interval of days or months after the initial injections(s) and the additional injections can prevent recurrence of the cancer.

The unitary daily dosage of EC0905 can vary significantly depending on the patient condition, the disease state being treated, the purity of the compounds and their route of administration and tissue distribution, and the possibility of co-usage of other therapeutic treatments, such as radiation therapy. The effective amount to be administered to a patient is based on body surface area, mass, and physician assessment of patient condition. Effective doses can range, for example, from about 1 ng/kg to about 1 mg/kg, from about 1 μg/kg to about 500 μg/kg, and from about 1 μg/kg to about 100 μg/kg. These doses are based on an average patient weight of about 70 kg, and the kg are kg of patient body weight (mass).

In one embodiment, the EC0905 conjugate can be administered in a dose of from about 1.0 ng/kg to about 1000 μg/kg, from about 10 ng/kg to about 1000 μg/kg, from about 50 ng/kg to about 1000 μg/kg, from about 100 ng/kg to about 1000 μg/kg, from about 500 ng/kg to about 1000 μg/kg, from about 1 ng/kg to about 500 μg/kg, from about 1 ng/kg to about 100 μg/kg, from about 1 μg/kg to about 50 μg/kg, from about 1 μg/kg to about 10 μg/kg, from about 5 μg/kg to about 500 μg/kg, from about 10 μg/kg to about 100 μg/kg, from about 20 μg/kg to about 200 μg/kg, from about 10 μg/kg to about 500 μg/kg, or from about 50 μg/kg to about 500 μg/kg. The total dose may be administered in single or divided doses and may, at the physician's discretion, fall outside of the typical range given herein. These dosages are based on an average patient weight of about 70 kg and the “kg” are kilograms of patient body weight. The physician will readily be able to determine doses for subjects whose weight falls outside this range, such as infants and the elderly.

In another embodiment, EC0905 can be administered in a dose of from about 1 μg/m² to about 500 m g/m², from about 1 μg/m² to about 300 mg/m², or from about 100 μg/m² to about 200 mg/m². In other embodiments, EC0905 can be administered in a dose of from about 1 mg/m² to about 500 mg/m², from about 1 mg/m² to about 300 mg/m², from about 1 mg/m² to about 200 mg/m², from about 1 mg/m² to about 100 mg/m², from about 1 mg/m² to about 50 mg/m², or from about 1 mg/m² to about 600 mg/m². The total dose may be administered in single or divided doses and may, at the physician's discretion, fall outside of the typical range given herein. These dosages are based on m² of body surface area.

The conjugates described herein may contain one or more chiral centers, or may otherwise be capable of existing as multiple stereoisomers. Accordingly, it is to be understood that the present invention includes pure stereoisomers as well as mixtures of stereoisomers, such as enantiomers, diastereomers, and enantiomerically or diastereomerically enriched mixtures. The conjugates described herein may be capable of existing as geometric isomers. Accordingly, it is to be understood that the present invention includes pure geometric isomers or mixtures of geometric isomers.

It is appreciated that the conjugate described herein may exist in unsolvated forms as well as solvated forms, including hydrated forms. In general, the solvated forms are equivalent to unsolvated forms and are encompassed within the scope of the present invention. The conjugate described herein may exist in multiple crystalline or amorphous forms. In general, all physical forms are equivalent for the methods, uses, kits, compounds, and compositions contemplated by the present invention and are intended to be within the scope of the present invention.

In another embodiment, compositions and/or dosage forms for administration of EC0905 are prepared from compounds with a purity of at least about 90%, or about 95%, or about 96%, or about 97%, or about 98%, or about 99%, or about 99.5%. In another embodiment, compositions and or dosage forms for administration of EC0905 are prepared from compounds with a purity of at least 90%, or 95%, or 96%, or 97%, or 98%, or 99%, or 99.5%.

As used herein, purity determinations may be based on weight percentage, mole percentage, and the like. In addition, purity determinations may be based on the absence or substantial absence of certain predetermined components, such as, but not limited to, folic acid, disulfide containing components not containing a vinca drug, oxidation products, disulfide components not containing a folate, and the like. It is also to be understood that purity determinations are applicable to solutions of the compounds and compositions prepared by the methods described herein. In those instances, purity measurements, including weight percentage and mole percentage measurements, are related to the components of the solution exclusive of the solvent. In another embodiment, EC0905 is provided in a sterile container or package. The purity of EC0905 may be measured using any conventional technique, including various chromatography or spectroscopic techniques, such as high pressure or high performance liquid chromatography (HPLC), nuclear magnetic resonance spectroscopy, TLC, UV absorbance spectroscopy, fluorescence spectroscopy, and the like.

In one embodiment of the above, the compound or composition is a multidose form. In another embodiment of the above, the compound or composition is a single dose form (i.e., a unit dose form or a dosage unit). One embodiment of the above dosage unit is one which provides on dilution or reconstitution with an aqueous diluent a solution comprising EC0905.

For purposes of this specification and appended claims, unless otherwise indicated, all numbers expressing quantities, percentages or proportions, and other numerical values used in the specification and claims, are understood as being modified in all instances by the term “about.” Accordingly, unless indicated to the contrary, the numerical parameters set forth in the following specification and attached claims are approximations that may vary depending upon the desired properties sought to be obtained by the present invention.

In another embodiment, the methods, compositions, pharmaceutical compositions, and kits, described herein include the following examples. The examples further illustrate additional features of the various embodiments of the invention described herein. However, it is to be understood that the examples are illustrative and are not to be construed as limiting other embodiments of the invention described herein. In addition, it is appreciated that other variations of the examples are included in the various embodiments of the invention described herein.

### EXAMPLES

### Example 1

### EC0905

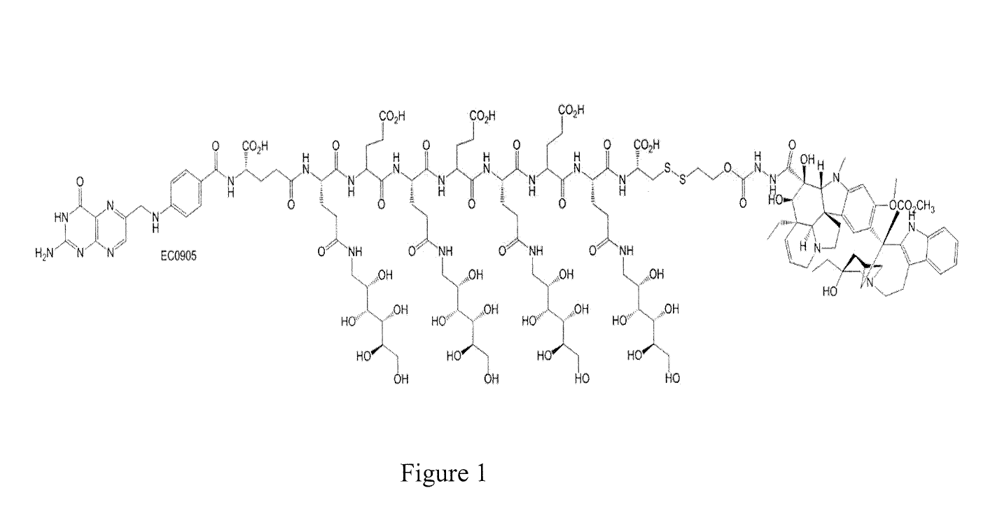

The structure of EC0905 is shown in FIG. 1. The carbohydrate-containing folic acid-spacer unit contains alternately repeating acidic (Glu) and saccharo-amino acids, thus providing high water-solubility of the final drug conjugate under physiological conditions (Vlahov et al., J. Org. Chern., 2010, 75, 3685-3691). This unit is assembled using standard fluorenylmethyloxycarbonyl-based solid phase peptide synthesis (Fmoc SPPS) on a Wang-resin. Desacetylvinblastine Hydrazide (DA VLBH), was prepared from commercially available vinblastine (VLB) sulfate (Barnett et al., J. Med. Chem., 1978, 21, 88). An activated carbonate (3) (Vlahov et al., Bioorg. & Medicinal Chem. Lett., 2006, 16, 5093) served as a heterobifunctional crosslinker to provide the drug-linker intermediate (4) for use in the assembly of the final conjugate. Treatment of a solution of folic acid-spacer in H20 under Argon and under extensive stirring with the Drug-Linker (4) unit resulted in a yellow suspension. According to the HPLC profile, the reaction was completed in 15 minutes. HPLC purification gave pure conjugate EC0905.

### Example 2

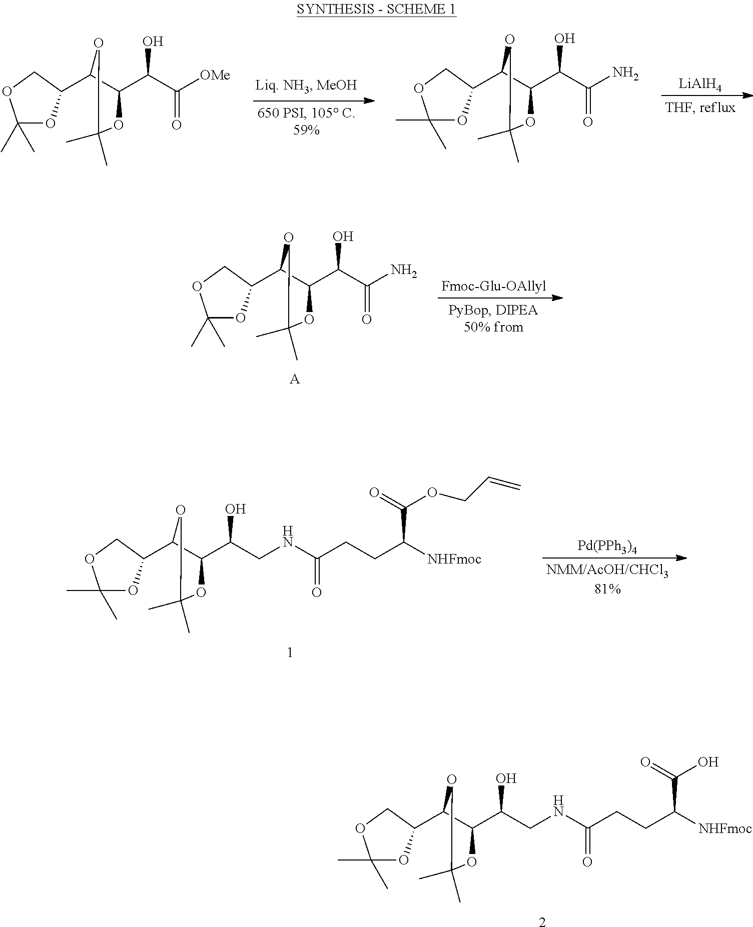

### Synthesis of 3,4;5,6-di-O-isopropylidene-1-amino-1-deoxy-(Fmoc-Glu-Oallyl)-D-glucitol (1)

Fmoc-Glu-OAll (2.17 g, 1 eq), PyBOP (2.88 g, 1 eq), and DIPEA (1.83 mL, 2 eq) were added to a solution of 3,4;5,6-di-O-isopropylidene-1-amino-1-deoxy-D-glucitol (A) (1.40 g, 5.3 mmol) in dry DMF (6 mL) and the reaction mixture was stirred at room temperature under Ar for 2 h. The solution was diluted with EtOAc (50 mL), washed with brine (10 mL×3), the organic layer separated, dried (MgSO₄), filtered, and concentrated to give a residue, which was purified by a flash column (silica gel, 60% EtOAc/petroleum ether) to afford the title compound (1.72 g, 50%) as a solid.

### Synthesis of 3,4;5,6-di-O-isopropylidene-1-amino-1-deoxy-(Fmoc-Glu-OH)-D-glucitol (2)

Pd(Ph₃)₄ (300 mg, 0.1 eq) was added to a solution of (1) (1.72 g, 2.81 mmol) in NMM/AcOH/CHCl₃ (2 mL/4 mL/74 mL). The resulting yellow solution was stirred at room temperature under Ar for 1 h, to which was added a second portion of Pd(Ph₃)₄ (300 mg, 0.1 eq). After stirring for an additional 1 h, the reaction mixture was washed with 1 N HCl (50 mL×3) and brine (50 mL), organic layer separated, dried (MgSO₄), filtered, and concentrated to give a yellow foamy solid, which was subject to chromatography (silica gel, 1% MeOH/CHCl₃ followed by 3.5% MeOH/CHCl₃) to give (2) (1.3 g, 81%) as a solid. Compound (A) may be obtained as outlined in the scheme and as described in WO 2009/002993 at pages 68 and 81-82.

### Synthesis of the Folate-Spacer Pte-γGlu-(Glu(1-amino-1-deoxy-D-glucitol)-Glu₃-Glu(1-amino-1-deoxy-D-glucitol)-Cys-OH

H-Cys(4-methoxytrityl)-2-chlorotrityl-resin (0.17 g, 0.10 mmol) was loaded into a peptide synthesis vessel and washed with i-PrOH (3×10 mL), followed by DMF (3×10 mL). To the vessel was introduced a solution of 3,4;5,6-di-O-isopropylidene-1-amino-1-deoxy(Fmoc-Glu-OH)-D-glucitol (0.13 mmol) in DMF, i-PrNEt (2 eq.), and PyBOP (1 eq.). The resulting solution was bubbled with Ar for 1 hr, the coupling solution was drained, and the resin washed with DMF (3×10 mL) and i-PrOH (3×10 mL). Kaiser tests were performed to assess reaction completion. Fmoc deprotection was carried out using 20% piperidine in DMF (3×10 mL). This procedure was repeated to complete all coupling steps (1.9 eq. of Fmoc-Glu(Ot-Bu)OH and Fmoc-Glu-Ot-Bu, and 1.6 eq. of N¹⁰TFA-pteroic acid were used on each of their respective coupling steps). After the pteroic acid coupling, the resin was washed with 2% hydrazine in DMF (3× for 5 min. each) to remove the trifluoroacetyl protecting group. The resin was washed with DMF (3×10 mL) and MeOH (10 mL) and dried under reduced pressure. The peptide was cleaved from the resin in the peptide synthesis vessel using a cleavage mixture consisting of 92.5% CF₃CO₂H, 2.5% H₂O, 2.5% triisopropylsilane, and 2.5% ethanedithiol. 25 mL of the cleavage mixture was added to the peptide synthesis vessel and the reaction was bubbled under Ar for 10 min. The resin was treated with two additional 15 mL quantities of the cleavage mixture for 5 minutes each. The cleavage mixture was concentrated to ca. 5 mL and ethyl ether was added to induce precipitation. The precipitate was collected by centrifugation, washed with ethyl ether 3 times, and dried under high vacuum, resulting in the recovery of ca. 100 mg of crude material. The compound was purified by prep. HPLC (mobile phase: A=10 mM ammonium acetate pH=5, B=ACN; method: 0% B to 20% B in 25 minutes at 15 mL/min). The pure fractions were pooled and freeze-dried, furnishing folate-spacer unit (51%).

### Example 3

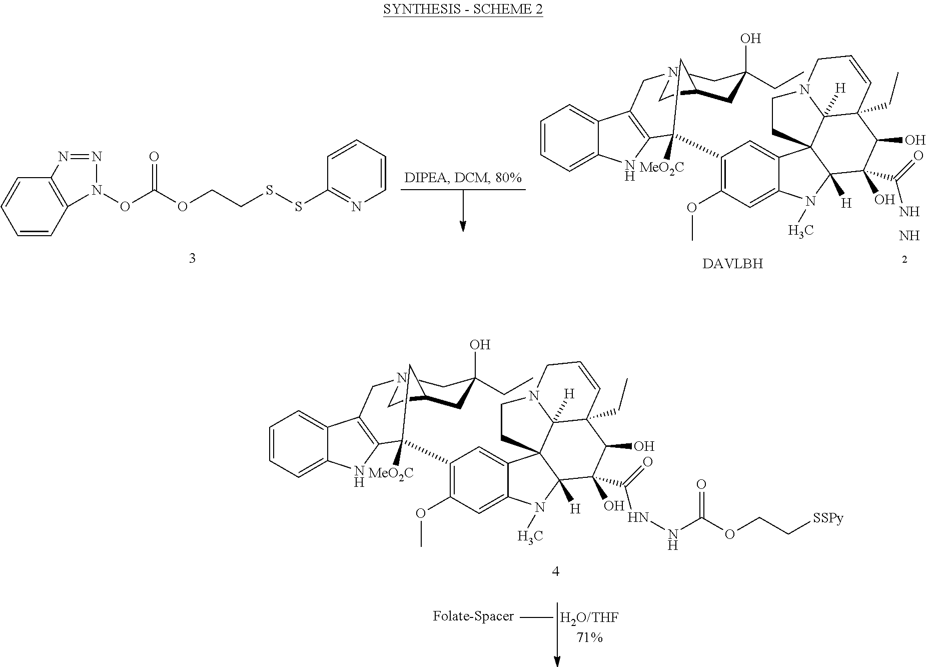

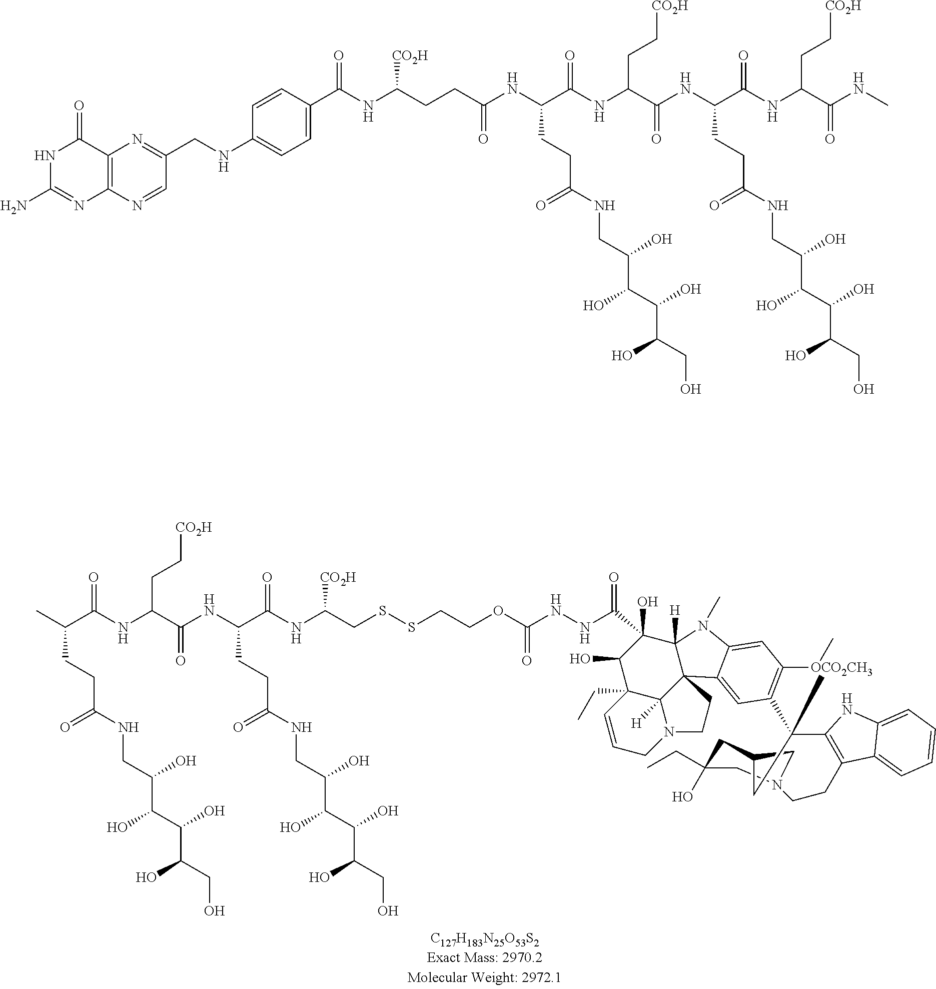

### Synthesis of EC905: Pte-γGlu-(Glu(I-amino-I-deoxy-D-glucitol)-Glu)₃-Glu(1-amino-1-deoxy-D-glucitol)-Cys(S-ethyl-3-(4 desacetylvinblastinyl)hydrazinecarboxylate)

In a polypropylene centrifuge bottle, the folate-spacer (0.015 mmol) was dissolved in 2.5 mL of Ar sparged water. In another flask, a saturated NaHCO₃ solution was Ar sparged for 10 min. The pH of the linker solution was carefully adjusted, with argon bubbling, to 6.9 using the NaHCO₃ solution. Vinblastine hydrazide-linker 4 (15 mg, 1.0 eq) in 2.5 mL of tetrahydrofuran (THF) was added quickly to the above solution. The resulting clear solution was stirred under argon. Progress of the reaction was monitored by analytical HPLC (2 mM sodium phosphate buffer, pH=7.0 and acetonitrile). After 20 min, 2 mM phosphate buffer (pH=7, 12 mL) was added to the reaction. The resulting cloudy solution was filtered and the filtrate was injected on the prep-HPLC (mobile phase: A=2 mM sodium phosphate pH=7, B=ACN; method: 1% B to 50% B in 25 minutes at 26 mL/min). Pure fractions were pooled and freeze-dried resulting in the recovery of EC0905 as a fluffy yellow powder (71%).

### Example 4

### Cell Growth Inhibition Studies

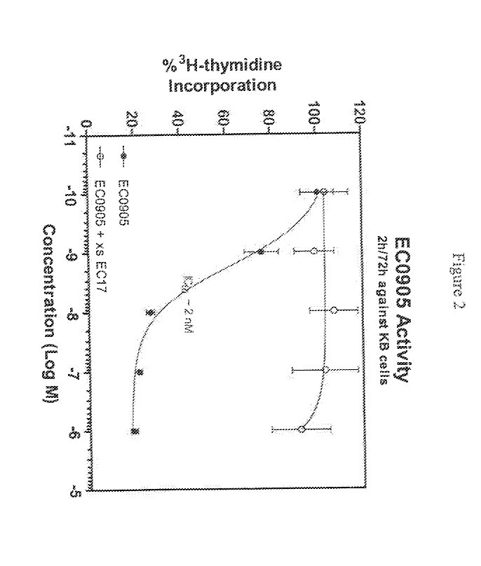

Folate receptor (FR)-positive human nasopharyngeal KB cells were grown continuously as a monolayer, using folate-free RPMI medium (FFRPMI) containing 10% heat-inactivated fetal calf serum (HIFCS) at 37° C. in a 5% CO₂/95% air-humidified atmosphere with no antibiotics. KB cells were seeded in 24-well plates 24 h before treatment with drugs and allowed to form nearly confluent monolayers. Cells were exposed to graded concentrations of EC0905 for 2 h at 37° C., rinsed 4 times with 0.5 mL of medium, and chased in 1 mL of fresh medium for 72 h. A total of five drug concentrations for each drug plus a drug-free control were evaluated. Cells were then treated with fresh medium containing ³H-thymidine for 2 h at 37° C. Cells were further washed with PBS and treated with ice-cold 5% trichloroacetic acid. After 15 min, the trichloroacetic acid was aspirated and the cells solubilized by the addition of 0.25 N sodium hydroxide for 15 min at room temperature. Each solubilized sample was transferred to scintillation vials containing Ecolume scintillation cocktail and counted in a liquid scintillation counter. Viability was assessed by measuring ³H-thymidine incorporation. Final results were expressed as the percentage of ³H-thymidine incorporation relative to untreated controls (FIG. 2). The results show that EC0905 inhibited ³H-thymidine incorporation in KB cells.

### Example 5

### In Vivo Antitumor Experiments

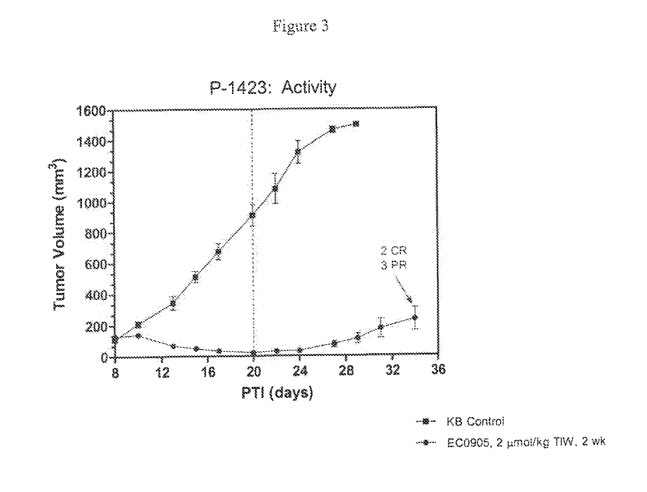

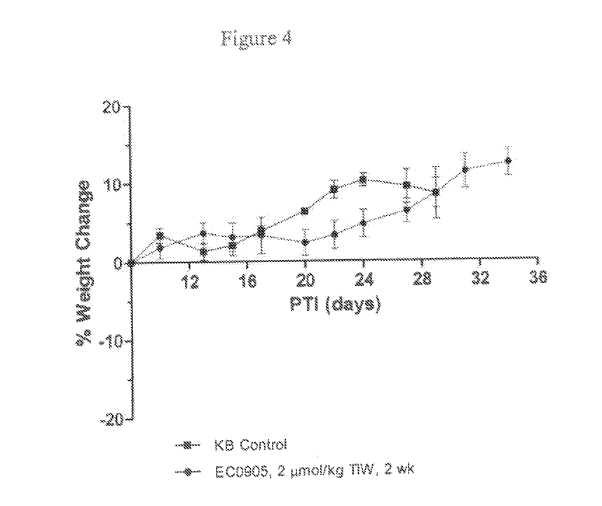

Four to six week-old female nu/nu mice (Charles River, Wilmington, Mass.) were maintained on a standard 12 h light-dark cycle and fed ad libitum with folate-deficient chow (Harlan diet #TD00434, Harlan Teklad, Madison, Wis.) for the duration of the experiment. KB cells (1×10⁶ per nu/nu mouse) in 100 μL were injected in the subcutis of the dorsal medial area. Mice were divided into groups of five, and test articles were freshly prepared and injected through the lateral tail vein under sterile conditions in a volume of 200 μL of phosphate-buffered saline (PBS). Intravenous (i.v.) treatments were typically initiated when the tumors were approximately 100-200 mm³ in volume. The mice in the control groups received no treatment. Growth of each subcutaneous tumor was followed post-tumor cell inoculation by measuring the tumor three times per week during treatment and twice per week thereafter until a volume of 1500 mm³ was reached. Tumors were measured in two perpendicular directions using Vernier calipers, and their volumes were calculated as 0.5×L×W², where L=measurement of longest axis in mm and W=measurement of axis perpendicular to L in mm (FIG. 3). As a general measure of toxicity, changes in body weights were determined on the same schedule as tumor volume measurements (FIG. 4). Survival of animals was monitored daily. All in vivo studies were performed in accordance with the American Accreditation Association of Laboratory Animal Care guidelines. For individual tumors, a partial response (PR) was defined as volume regression>50% but with measurable tumor (>2 mm³) remaining at all times. Complete response (CR) was defined as a disappearance of measurable tumor mass (<2 mm³) at some point until the end of the study. The results show that EC0905 inhibited tumor growth in the mice.

What is claimed is:

1. A compound of the formula 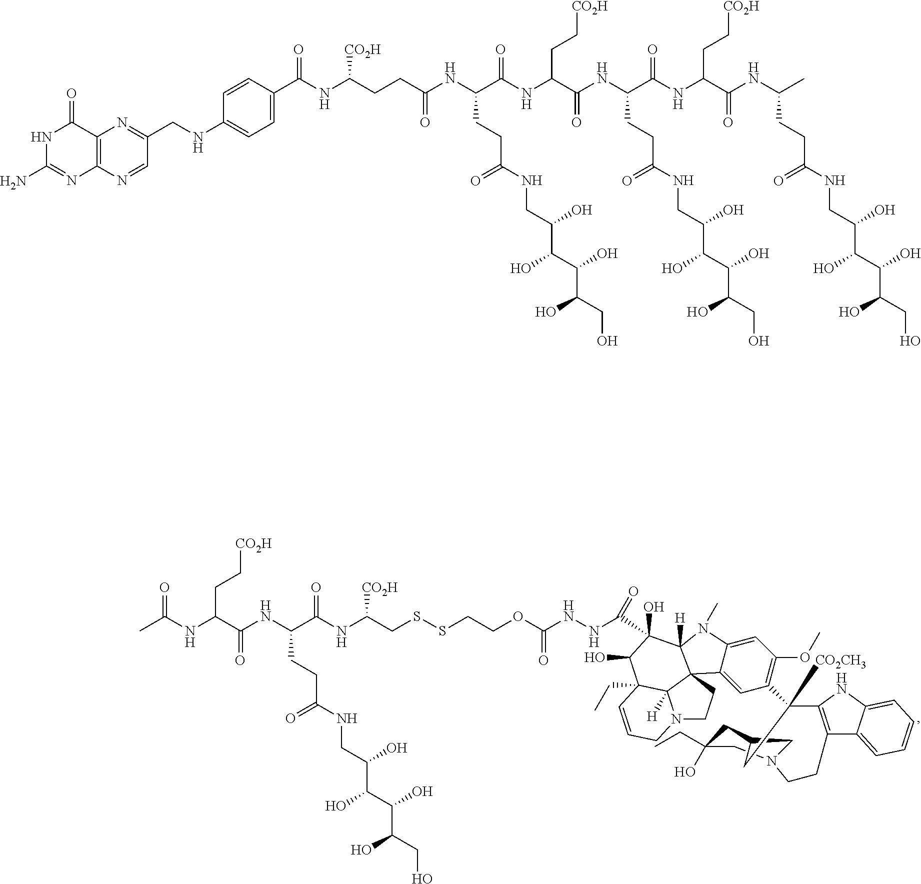
or a pharmaceutically acceptable salt, solvate, or hydrate thereof.

2. A composition comprising the compound of claim 1, wherein the composition further comprises a pharmaceutically acceptable carrier.

3. The composition of claim 2, wherein the composition is an inhalation dosage form, an oral dosage form, or a parenteral dosage form.

4. The composition of claim 3, wherein the composition is a parenteral dosage form.

5. The composition of claim 4, wherein the parenteral dosage form is an intradermal dosage form, a subcutaneous dosage form, an intramuscular dosage form, an intraperitoneal dosage form, an intravenous dosage form, or an intrathecal dosage form.

6. The composition of claim 2, wherein the pharmaceutically acceptable carrier comprises a liquid carrier.

7. The composition of claim 6, wherein the liquid carrier is saline, glucose, an alcohol, a glycol, an ester, an amide, or a combination thereof.

8. The composition of claim 2, wherein the compound is in the form of a reconstitutable lyophilizate.

9. The composition of claim 2, wherein the compound is in the form of a lyophilizate.

10. The composition of claim 2, wherein the compound is in the form of a solid.

11. The composition of claim 2, wherein the purity of the compound is at least 90% based on weight percent.

12. The composition of claim 2, wherein the purity of the compound is at least 95% based on weight percent.

13. The composition of claim 2, wherein the purity of the compound is at least 98% based on weight percent.

14. The composition of claim 2, wherein the purity of the compound is at least 99% based on weight percent.

15. A kit comprising a sterile vial, the composition of claim 2, and instructions for use describing use of the composition for treating a patient with cancer.

16. The kit of claim 15, wherein the composition is in the form of a reconstitutable lyophilizate.

17. The kit of claim 15, wherein the dose of the composition is in the range of 1 to 5 μg/kg of patient body weight.

18. The kit of claim 15, wherein the dose of the composition is in the range of 1 to 3 μg/kg of patient body weight.

19. The kit of claim 15, wherein the purity of the composition is at least 90% based on weight percent.

20. The kit of claim 15, wherein the purity of the composition is at least 95% based on weight percent.

21. The kit of claim 15, wherein the purity of the composition is at least 98% based on weight percent.

22. The kit of claim 15, wherein the purity of the composition is at least 99% based on weight percent.

23. The kit of claim 15, wherein the composition is in a parenteral dosage form.

24. The kit of claim 23, wherein the parenteral dosage form is an intradermal dosage form, a subcutaneous dosage form, an intramuscular dosage form, an intraperitoneal dosage form, an intravenous dosage form, or an intrathecal dosage form.

25. The kit of claim 15, wherein the composition further comprises a pharmaceutically acceptable carrier.

26. The kit of claim 25, wherein the pharmaceutically acceptable carrier is a liquid carrier, wherein the liquid is saline, glucose, an alcohol, a glycol, an ester, an amide, or a combination thereof.

27. The composition of claim 2, wherein the pharmaceutically acceptable salt of EC0905 is a sodium salt.
